(19) United States
(12) Patent Application Publication
(10) Pub. No.: US 2023/0059938 A1
(43) Pub. Date: Feb. 23, 2023

(54) PHARMACEUTICAL FORMULATION FOR INHIBITING BODY MALODOUR

(71) Applicant: National Skin Centre (Singapore) Pte Ltd, Singapore (SG)

(72) Inventors: Hong Liang TEY, Singapore (SG); Winnie CHOO, Singapore (SG)

(73) Assignee: National Skin Centre (Singapore) Pte Ltd, Singapore (SG)

(21) Appl. No.: 17/784,389

(22) PCT Filed: Dec. 9, 2020
(86) PCT No.: PCT/SG2020/050730

§ 371 Date: Jun. 10, 2022

(30) Foreign Application Priority Data
Dec. 12, 2019 (SG) 10201912063P

(51) Int. Cl. A61K 31/40; A61K 47/10; A61P 17/00; A61K 9/00

(52) U.S. Cl. CPC A61K 31/40; A61K 47/10; A61P 17/00; A61K 9/0014; A61K 9/0017

(57) ABSTRACT

There is provided a pharmaceutical formulation comprising an anticholinergic agent and a ternary solvent comprising 5% to 15% v/v glycol, 60% to 70% v/v alcohol and 5% to 20% v/v water. There is also provided a method of preparing the pharmaceutical formulation as disclosed herein, comprising the step of mixing the anticholinergic agent to a mixture containing alcohol, glycol, water, humectant and pH buffering agent at room temperature. There is also provided a method of inhibiting non-pathological body malodour in a mammal. There is also provided a method of treating apocrine secretion causing odour and reduction of microbial overgrowth in a mammalian subject, and uses of a pharmaceutical formulation.

### TECHNICAL FIELD

The present invention relates to a pharmaceutical formulation, a method of preparing the same, and uses thereof

### BACKGROUND ART

Axillary bromhidrosis, or commonly known as “armpit/underarm odour”, is a very common problem in adults and results in severe social embarrassment and dysfunction. The link between body odour, the axillary apocrine gland, and underarm bacteria was first recognised in the 1950s. Secretion from the apocrine glands that becomes malodourous on bacterial breakdown is the main cause of bromhidrosis.

In humans, apocrine sweat glands are found only in certain locations of the body: the axillae, areola and nipples of the breast, ear canal, eyelids, wings of the nostril, perianal region, and some parts of the external genitalia. Apocrine glands are located in the lower part of the reticular dermis and the subcutaneous tissue. Men have more apocrine glands than women. Racial differences in body odour have been related to differences in the number of apocrine glands in the various races. The exact mechanism of stimulation of the human apocrine gland is not defined but present evidence suggests this is due to catecholamines, and the cholinergic sympathetic nervous system indirectly affects this process. In humans, apocrine glands are predominantly located in the axillae and axillary bromhidrosis is the predominant contributor to body odour.

The pathogenesis of apocrine bromhidrosis is twofold; secretion from the apocrine glands followed by breakdown of the secretion by resident bacteria to produce unsaturated fatty acids, especially trans-3-methyl-2-hexenoic acid, isovaleric acid and propionic acid, liberates unpleasant smells. Current treatment options, especially topical applications, are limited and poor. The most common topical applications include deodorants, topical antibiotics and antiseptics, and anti-perspirants containing aluminium salts. Deodorants mask the body odour but they do not stop the smell. The fragrances found in the deodorants often cause contact dermatitis. Topical antibiotics and antiseptics, including antiseptic soaps, help limit the growth of the contributory bacteria that decompose the apocrine secretions. However, these are often ineffective or the effects are short-lived. Anti-perspirants containing aluminium salts inhibit sweating by forming a temporary plug in the sweat pores. Although this is currently the best topical option, it frequently causes inflammation around the blocked sweat pores, resulting in an itchy rash. Anti-perspirants also stains clothing and do not work in a significant number of people. There are non-topical treatment options available, but they are either inconvenient (iontophoresis) or invasive and expensive (botulinium toxin, lasers, microwave destruction and surgery).

As a result of the deficiencies in the available therapies, many people are not receiving treatment for bromhidrosis. Therefore, there is a need to provide a novel treatment, in the form of a pharmaceutical formulation and a method of preparing the same that overcome or ameliorate one or more of the disadvantages mentioned above.

In smaller mammals such as cats and dogs, apocrine glands are widely distributed over their bodies as they are associated with every hair follicle. These apocrine glands have the capability to produce sweat when sympathetic and/or parasympathetic nervous system innervated. at those glands are stimulated but not so much with emotional or thermal stimuli. This secretion is also prone to bacterial breakdown and release of malodourous smell. This tends to happen at the skin folds of the animals where the skin folds near the limbs or neck tend to trap heat, moist and bacteria.

This pharmaceutical formulation is useful to reduce the general unpleasant smell at the skin folds of the animals. It is not to treat infection of the ceruminous glands of the ears nor circumanal glands of the anal sacs.

### SUMMARY

The present disclosure relates to a pharmaceutical formulation comprising an anticholinergic agent and a ternary solvent comprising 5% to 15% v/v glycol, 60% to 70% v/v alcohol and 5% to 20% v/v water.

In one aspect, the present disclosure relates to a pharmaceutical formulation comprising glycopyrronium bromide and a ternary solvent comprising 5% to 15% v/v propylene glycol, 60% to 70% v/v isopropanol and 5% to 20% v/v water.

In another aspect, the present disclosure relates to a pharmaceutical formulation consisting essentially of glycopyrronium bromide and a ternary solvent comprising 5% to 15% v/v propylene glycol, 60% to 70% v/v isopropanol and 5% to 20% v/v water.

Advantageously, the pharmaceutical formulation may effectively target the two steps involving bromhidrosis, wherein the anticholinergic agent inhibits apocrine secretion and the high concentration of alcohol in the ternary solvent acts as a broad-spectrum antimicrobial agent limiting the growth of the contributory bacteria decomposing the apocrine secretions.

When sweat macerates the stratum corneum in skin fold regions such as the axillae, swelling and development of pools of water in the intercellular region can lead to narrowing of the infundibular region of hair follicles. This results in impediment of skin permeation of the anticholinergic agent via the hair follicular route. Further advantageously, the pharmaceutical formulation may circumvent this problem of reduced bioavailability of the hydrophilic anticholinergic agent, through enhancing skin permeability of the anticholinergic agent via the transcellular and intercellular pathways.

Still further advantageously, the pharmaceutical formulation may not cause blockage of the sweat pores which can result in inflammation around the sweat ducts.

Still further advantageously, the pharmaceutical formulation may be readily applied onto the skin and is rapidly dried after application.

The present disclosure relates to a method of preparing the pharmaceutical formulation as disclosed herein, comprising the step of mixing the anticholinergic agent to a mixture containing alcohol, glycol, water, humectant and pH buffering agent at room temperature.

In another aspect, the present disclosure relates to a method of preparing the pharmaceutical formulation as disclosed herein, comprising the step of mixing the glycopyrronium bromide to a mixture containing isopropanol, propylene glycol, water, humectant and pH buffering agent at room temperature.

The present disclosure relates to a method of inhibiting non-pathological body malodour in a mammal, comprising the step of administrating a pharmaceutical formulation to an area on said mammal, to inhibit apocrine secretion and inhibit the activity or prevent the overgrowth of the microorganism resident on said area, wherein said pharmaceutical formulation comprises an anticholinergic agent and a ternary solvent comprising 5% to 15% v/v glycol, 60% to 70% v/v alcohol and 5% to 20% v/v water.

In another aspect, the present disclosure relates to a method of inhibiting non-pathological body malodour in a mammal, comprising the step of administrating a pharmaceutical formulation to an area on said mammal, to inhibit apocrine secretion and inhibit the activity or prevent the overgrowth of the microorganism resident on said area, wherein said pharmaceutical formulation comprises glycopyrronium bromide and a ternary solvent comprising 5% to 15% v/v glycol, 60% to 70% v/v isopropanol and 5% to 20% v/v water.

In another aspect, the present disclosure relates to a method of inhibiting non-pathological body malodour in a mammal, comprising the step of administrating a pharmaceutical formulation to an area on said mammal, to inhibit apocrine secretion and inhibit the activity or prevent the overgrowth of the microorganism resident on said area, wherein said pharmaceutical formulation consists essentially of glycopyrronium bromide and a ternary solvent comprising 5% to 15% v/v propylene glycol, 60% to 70% v/v isopropanol and 5% to 20% v/v water.

Advantageously, the method of inhibiting non-pathological body malodour may result in about 63.0%, about 64.0%, about 65.0%, about 66.0%, about 67.0%, about 68.0%, about 69.0%, about 70.0%, about 71.0%, about 72.0%, or about 73.0% mean reduction in axillary body odour in human subjects.

Further advantageously, the method of inhibiting non-pathological body malodour may result in about 53.0%, about 54.0%, about 55.0%, about 56.0%, about 57.0%, about 58.0%, about 59.0%, about 60.0%, about 61.0%, about 62.0%, or about 63.0% mean reduction in reduction in axillary body odour in dog subjects.

The present disclosure relates to a method of treating apocrine secretion causing odour and reduction of microbial overgrowth in a mammalian subject, comprising administrating a pharmaceutical formulation to an area on said mammalian subject exhibiting said microbial overgrowth, wherein said pharmaceutical formulation comprises an anticholinergic agent and a ternary solvent comprising 5% to 15% v/v glycol, 60% to 70% v/v alcohol and 5% to 20% v/v water.

In another aspect, the present disclosure relates to a method of treating apocrine secretion causing odour and reduction of microbial overgrowth in a mammalian subject, comprising administrating the pharmaceutical formulation to an area on said mammalian subject exhibiting said microbial overgrowth, wherein said pharmaceutical formulation comprises glycopyrronium bromide and a ternary solvent comprising 5% to 15% v/v propylene glycol, 60% to 70% isopropanol and 5% to 20% v/v water.

The present disclosure relates to a pharmaceutical formulation for use in the treatment of apocrine secretion causing odour and reduction of microbial overgrowth in a mammalian subject, wherein said pharmaceutical formulation comprises an anticholinergic agent and a ternary solvent comprising 5% to 15% v/v glycol, 60% to 70% v/v alcohol and 5% to 20% v/v water.

In another aspect, the present disclosure relates to a pharmaceutical formulation for use in the treatment of apocrine secretion causing odour and reduction of microbial overgrowth in a mammalian subject, wherein said pharmaceutical formulation comprises glycopyrronium bromide and a ternary solvent comprising 5% to 15% v/v propylene glycol, 60% to 70% isopropanol and 5% to 20% v/v water.

The present disclosure relates to use of a pharmaceutical formulation, in the manufacture of a medicament for the treatment of apocrine secretion causing odour and reduction of microbial overgrowth in a mammalian subject, wherein said pharmaceutical formulation comprises an anticholinergic agent and a ternary solvent comprising 5% to 15% v/v glycol, 60% to 70% v/v alcohol and 5% to 20% v/v water.

In another aspect, the present disclosure relates to use of a pharmaceutical formulation, in the manufacture of a medicament for the treatment of apocrine secretion causing odour and reduction of microbial overgrowth in a mammalian subject, wherein said pharmaceutical formulation comprises glycopyrronium bromide and a ternary solvent comprising 5% to 15% v/v propylene glycol, 60% to 70% isopropanol and 5% to 20% v/v water.

### Definitions

The following words and terms used herein shall have the meaning indicated:

The term “anticholinergic” as used herein refers to the capability of a substance to block the binding of the neurotransmitter acetylcholine to acetylcholine receptors on multiple sites of the body including sweat glands.

The term “bromhidrosis” as used herein refers to a condition of abnormal or offensive body odour, due largely to microbial breakdown of the apocrine gland secretion.

The term “antimicrobial” as used herein refers to the capability to cause cell inhibition, cell injury, cell death or to control the growth of target bacteria and fungi microorganisms.

The term “ternary solvent” as used herein refers to a mixture system composed of three different component liquids that is capable of dissolving the anticholinergic agent.

The term “mammals” as used herein refers to vertebrate animals characterized by the presence of mammary glands which produce milk for nursing their young, a neocortex, fur or hair, three middle ear bones and four-chambered heart.

The term “topical” as used herein refers to application on body surfaces.

The term “non-pathological” as used herein refers to not relating to nor indicative of nor caused by pathology or disease.

The term “malodour” as used herein refers to odour that is offensively unpleasant.

Unless specified otherwise, the terms “comprising” and “comprise”, and grammatical variants thereof, are intended to represent “open” or “inclusive” language such that they include recited elements but also permit inclusion of additional, unrecited elements.

As used herein, the term “about”, in the context of concentrations of components of the formulations, typically means +/−5% of the stated value, more typically +/−4% of the stated value, more typically +/−3% of the stated value, more typically, +/−2% of the stated value, even more typically +/−1% of the stated value, and even more typically +/−0.5% of the stated value.

Throughout this disclosure, certain embodiments may be disclosed in a range format. It should be understood that the description in range format is merely for convenience and brevity and should not be construed as an inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Certain embodiments may also be described broadly and generically herein. Each of the narrower species and sub-generic groupings falling within the generic disclosure also form part of the disclosure. This includes the generic description of the embodiments with a proviso or negative limitation removing any subject matter from the genus, regardless of whether or not the excised material is specifically recited herein.

Detailed Disclosure of Embodiments

Exemplary, non-limiting embodiments of a pharmaceutical formulation will now be disclosed.

The pharmaceutical formulation comprising an anticholinergic agent and a ternary solvent comprising 5% to 15% v/v glycol, 60% to 70% v/v alcohol and 5% to 20% v/v water.

The pharmaceutical formulation consisting of an anticholinergic agent and a ternary solvent consisting of 5% to 15% v/v glycol, 60% to 70% v/v alcohol and 5% to 20% v/v water.

The pharmaceutical formulation consisting essentially of an anticholinergic agent and a ternary solvent consisting essentially of 5% to 15% v/v glycol, 60% to 70% v/v alcohol and 5% to 20% v/v water.

When sweat macerates the stratum corneum in skin fold regions such as the axillae, swelling and development of pools of water in the intercellular region can lead to narrowing of the infundibular region of hair follicles. This results in impediment of skin permeation of the anticholinergic agent via the hair follicular route. The pharmaceutical formulation may circumvent this problem of reduced bioavailability of the hydrophilic anticholinergic agent, through enhancing skin permeability of the anticholinergic agent via the transcellular and intercellular pathways.

The anticholinergic agent may be selected from the group consisting of glycopyrronium bromide, hyoscyamine, atropine, scopolamine, benzatropine, clidinium bromide, cyclopentolate, darifenacin, dicylomine, fesoterodine, homatropine hydrobromide, ipratropium, orphenadrine, oxybutynin, propantheline, methscopolamine, solifenacin, tiotropium, tolterodine, trihexphenidyl, trospium and mixtures thereof.

The alcohol may be selected from the group consisting of ethanol and isopropanol.

The glycol may be selected from the group consisting of propylene glycol, butylene glycol and pentylene glycol.

The anticholinergic agent may be in an amount of about 1.0% w/v, about 2.0% w/v, about 3.0% w/v, about 4.0% w/v, about 5.0% w/v or about 6.0% w/v of the pharmaceutical formulation.

The concentrations of the components in the ternary solvent may be as follow: glycol at about 5.0% v/v, about 6.0% v/v, about 7.0% v/v, about 8.0% v/v, about 9.0% v/v, about 10.0% v/v, about 11.0% v/v, about 12.0% v/v, about 13.0% v/v, about 14.0% v/v or about 15.0% v/v of the pharmaceutical formulation; alcohol at about 60.0% v/v, about 61.0% v/v, about 62.0% v/v, about 63.0% v/v, about 64.0% v/v, about 65.0% v/v, about 66.0% v/v, about 67.0% v/v, about 68.0% v/v, about 69.0% v/v or about 70.0% v/v of the pharmaceutical formulation; and water at about 5.0% v/v, about 6.0% v/v, about 7.0% v/v, about 8.0% v/v, about 9.0% v/v, about 10.0% v/v, about 11.0% v/v, about 12.0% v/v, about 13.0% v/v, about 14.0% v/v, about 15% v/v, about 16% v/v, about 17% v/v, about 18% v/v, about 19% v/v or about 20.0% v/v of the pharmaceutical formulation.

When the anticholinergic agent is glycopyrronium bromide and the alcohol is isopropanol, the ratio of the components in the ternary solvent may be such that the ratio of propylene glycol:isopropanol: water is 2: 13: 2.5 v/v.

The pharmaceutical formulation may further comprise an excipient material suitable for topical administration.

The excipient material may be selected from the group consisting of anti-bacterials, anti-fungals, humectants, emulsifiers, preservatives, dispersants, emollients, surfactants, structurants, absorption promoters, antiseptics, anaesthetics, keratolytics, wound healing agents, lubricants, anti-perspirants, depilatories, UV-protectants, anti-inflammatories, steroids, antioxidants, antihistamines, skin and hair conditioners, fragrances, essential oils and natural plant extracts.

The humectant may selected from the group consisting of glycerine, glycerol, lecithin, gelatin, lactic acid, hyaluronic acid, glyceryl triacetate, hexylene glycol, butylene glycol, sorbitol, allantoin, sodium hyaluronate, sodium lactate, ammonium lactate, sodium pyrrolidine and urea.

The pharmaceutical forumulation may be for topical use.

The pharmaceutical formulation may be anticholinergic and antimicrobial.

The pharmaceutical formulation may be anticholinergic and antibacterial.

The pharmaceutical formulation may be in the form selected from the group consisting of solution, lotion, cream, ointment, gel, paste, aerosol, foam and spray.

When the pharmaceutical formulation is in the form of a spray for use in human, the pharmaceutical formulation may be sprayed on the axilla 1 or 2 sprays per administration at a frequency of at least once in a day, wherein once in the morning and optionally once in the afternoon, depending on a person's requirement.

When the pharmaceutical formulation is in the form of a spray for use in dog, the pharmaceutical formulation may be sprayed on the affected skin at a frequency of 1 to 3 times a day and 4 to 8 hours between sprays.

The amount of anticholinergic agent in each spray administered may be about 2.00 mg, about 2.10 mg, about 2.20 mg, about 2.30 mg, about 2.40 mg, about 2.50 mg, about 2.60 mg, about 2.70 mg or about 2.80 mg.

Exemplary, non-limiting embodiments of a method of preparing the pharmaceutical formulation will now be disclosed.

The method of preparing the pharmaceutical formulation as disclosed herein, comprising the step of mixing the anticholinergic agent to a mixture containing alcohol, glycol, water, humectant and pH buffering agent at room temperature.

The alcohol may be selected from the group consisting of ethanol and isopropanol.

The pH buffering agent may be selected from the group consisting of organic acids, amino acids, polyaminocarboxylic acids, citrate salts, polyphosphates and combinations thereof.

The pH buffering agent may be selected from the group consisting of citric acid, acetic acid, tartaric acid, ethylenediaminetetraacetic acid, 2,3-dimercaptopropanesulfonic acid, thiamine tetrahydrofurfuryl acid, alpha lipoic acid, glycine, sodium citrate, potassium citrate, sodium phosphate and combinations thereof.

The humectant may be in an amount of about 5.0% w/v, about 6.0% w/v, about 7.0% w/v, about 8.0% w/v, about 9.0% w/v, about 10.0% w/v, about 11.0% w/v, about 12.0% w/v, about 13.0% w/v, about 14.0% w/v, about 15.0% w/v, about 16.0% w/v, about 17.0% w/v, about 18.0% w/v, about 19.0% w/v or about 20.0% w/v of the pharmaceutical formulation..

The pH buffering agent may be in an amount in the range of about 0.05% w/v to about 1.00% w/v, about 0.10% w/v to about 1.00% w/v, about 0.20% w/v to about 1.00% w/v, about 0.40% w/v to about 1.00% w/v, about 0.60% w/v to about 1.00% w/v, about 0.80% w/v to about 1.00% w/v, about 0.05% w/v to about 0.10% w/v, about 0.05% w/v to about 0.20% w/v, about 0.05% w/v to about 0.40% w/v, about 0.05% w/v to about 0.60% w/v, about 0.05% w/v to about 0.80% w/v, about 0.10% w/v to about 0.20% w/v, about 0.10% w/v to about 0.40% w/v, about 0.10% w/v to about 0.60% w/v, about 0.10% w/v to about 0.80% w/v, about 0.20% w/v to about 0.40% w/v, about 0.20% w/v to about 0.60% w/v, about 0.20% w/v to about 0.80% w/v, about 0.40% w/v to about 0.60% w/v, about 0.40% w/v to about 0.80% w/v, or about 0.60% w/v to about 0.80% w/v of the pharmaceutical formulation.

Exemplary, non-limiting embodiments of a method of inhibiting a non-pathological body malodour and a method of treating apocrine secretion causing odour and reduction of microbial overgrowth in a mammalian subject will now be disclosed.

There is provided a method of inhibiting non-pathological body malodour in a mammal, comprising the step of administrating a pharmaceutical formulation to an area on said mammal, to inhibit apocrine secretion and inhibit the activity or prevent the overgrowth of the microorganism resident on said area, wherein said pharmaceutical formulation comprises an anticholinergic agent and a ternary solvent comprising 5% to 15% v/v glycol, 60% to 70% v/v alcohol and 5% to 20% v/v water.

There is also provided a method of inhibiting non-pathological body malodour in a mammal, comprising the step of administrating a pharmaceutical formulation to an area on said mammal, to inhibit apocrine secretion and inhibit the activity or prevent the overgrowth of the microorganism resident on said area, wherein said pharmaceutical formulation consists of an anticholinergic agent and a ternary solvent consisting of 5% to 15% v/v glycol, 60% to 70% v/v alcohol and 5% to 20% v/v water.

There is also provided a method of inhibiting non-pathological body malodour in a mammal, comprising the step of administrating a pharmaceutical formulation to an area on said mammal, to inhibit apocrine secretion and inhibit the activity or prevent the overgrowth of the microorganism resident on said area, wherein said pharmaceutical formulation consists essentially of an anticholinergic agent and a ternary solvent consisting essentially of 5% to 15% v/v glycol, 60% to 70% v/v alcohol and 5% to 20% v/v water.

When the body malodour in a mammal is non-pathological, the overgrowth of microorganism may be defined as below 10⁵ organisms per gram of tissue or per mL of biological fluid.

The method of inhibiting non-pathological body malodour may result in about 63.0%, about 64.0%, about 65.0%, about 66.0%, about 67.0%, about 68.0%, about 69.0%, about 70.0%, about 71.0%, about 72.0%, or about 73.0% mean reduction in axillary body odour in human subjects.

The method of inhibiting non-pathological body malodour may result in about 53.0%, about 54.0%, about 55.0%, about 56.0%, about 57.0%, about 58.0%, about 59.0%, about 60.0%, about 61.0%, about 62.0%, or about 63.0% mean reduction in reduction in axillary body odour in dog subjects.

The mammal may be selected from the group consisting of primates, caniformia and artiodactyla.

The microorganism may selected from the group consisting of Corynebacterium spp., Corynebacterium minutissimum, Corynebacterium striatum, Corynebacterium jeikeium, Staphylococcus spp., Staphylococcus aureus, Staphylococcus epidermidis, Staphylococcus haemolyticus, Staphylococcus pseudintermedius, Streptococcus spp., Micrococcus spp, Propionbacterium spp., Propionbacterium acnes, Anaerococcus spp., Candida spp., Escherichia coli, Malassezia pachydermatis and Sphingomonas paucimobilis.

There is also provided a method of treating apocrine secretion causing odour and reduction of microbial overgrowth in a mammalian subject, comprising administrating a pharmaceutical formulation to an area on said mammalian subject exhibiting said microbial overgrowth, wherein said pharmaceutical formulation comprises an anticholinergic agent and a ternary solvent comprising 5% to 15% v/v glycol, 60% to 70% v/v alcohol and 5% to 20% v/v water.

There is also provided a method of treating apocrine secretion causing odour and reduction of microbial overgrowth in a mammalian subject, comprising administrating a pharmaceutical formulation to an area on said mammalian subject exhibiting said microbial overgrowth, wherein said pharmaceutical formulation consists of an anticholinergic agent and a ternary solvent consisting of 5% to 15% v/v glycol, 60% to 70% v/v alcohol and 5% to 20% v/v water.

There is also provided a method of treating apocrine secretion causing odour and reduction of microbial overgrowth in a mammalian subject, comprising administrating a pharmaceutical formulation to an area on said mammalian subject exhibiting said microbial overgrowth, wherein said pharmaceutical formulation consists essentially of an anticholinergic agent and a ternary solvent consisting essentially of 5% to 15% v/v glycol, 60% to 70% v/v alcohol and 5% to 20% v/v water.

The overgrowth of microorganism may be defined as more than 10⁵ organisms per gram of tissue or per mL of biological fluid.

The mammalian subject may be selected from the group consisting of primates, caniformia and artiodactyla.

The microbial overgrowth may be caused by a microorganism selected from the group consisting of Corynebacterium spp., Corynebacterium minutissimum, Corynebacterium striatum, Corynebacterium jeikeium, Staphylococcus spp., Staphylococcus aureus, Staphylococcus epidermidis, Staphylococcus haemolyticus, Staphylococcus pseudintermedius, Streptococcus spp., Micrococcus spp, Propionbacterium spp., Propionbacterium acnes, Anaerococcus spp., Candida spp., Escherichia coli, Malassezia pachydermatis and Sphingomonas paucimobilis.

Exemplary, non-limiting embodiments of uses of a pharmaceutical formulation will now be disclosed.

There is provided a pharmaceutical formulation for use in the treatment of apocrine secretion causing odour and reduction of microbial overgrowth in a mammalian subject, wherein said pharmaceutical formulation comprises an anticholinergic agent and a ternary solvent comprising 5% to 15% v/v glycol, 60% to 70% v/v alcohol and 5% to 20% v/v water.

There is provided a pharmaceutical formulation for use in the treatment of apocrine secretion causing odour and reduction of microbial overgrowth in a mammalian subject, wherein said pharmaceutical formulation consists of an anticholinergic agent and a ternary solvent consisting of 5% to 15% v/v glycol, 60% to 70% v/v alcohol and 5% to 20% v/v water.

There is provided a pharmaceutical formulation for use in the treatment of apocrine secretion causing odour and reduction of microbial overgrowth in a mammalian subject, wherein said pharmaceutical formulation consists essentially of an anticholinergic agent and a ternary solvent consisting essentially of 5% to 15% v/v glycol, 60% to 70% v/v alcohol and 5% to 20% v/v water.

There is also provided use of a pharmaceutical formulation, in the manufacture of a medicament for the treatment of apocrine secretion causing odour and reduction of microbial overgrowth in a mammalian subject, wherein said pharmaceutical formulation comprises an anticholinergic agent and a ternary solvent comprising 5% to 15% v/v glycol, 60% to 70% v/v alcohol and 5% to 20% v/v water.

There is also provided use of a pharmaceutical formulation, in the manufacture of a medicament for the treatment of apocrine secretion causing odour and reduction of microbial overgrowth in a mammalian subject, wherein said pharmaceutical formulation consists of an anticholinergic agent and a ternary solvent consisting of 5% to 15% v/v glycol, 60% to 70% v/v alcohol and 5% to 20% v/v water.

There is also provided use of a pharmaceutical formulation, in the manufacture of a medicament for the treatment of apocrine secretion causing odour and reduction of microbial overgrowth in a mammalian subject, wherein said pharmaceutical formulation consists essentially of an anticholinergic agent and a ternary solvent consisting essentially of 5% to 15% v/v glycol, 60% to 70% v/v alcohol and 5% to 20% v/v water.

The mammalian subject may be selected from the group consisting of primates, caniformia and artiodactyla.

The microbial overgrowth may be caused by a microorganism selected from the group consisting of Corynebacterium spp., Corynebacterium minutissimum, Corynebacterium striatum, Corynebacterium jeikeium, Staphylococcus spp., Staphylococcus aureus, Staphylococcus epidermidis, Staphylococcus haemolyticus, Staphylococcus pseudintermedius, Streptococcus spp., Micrococcus spp, Propionbacterium spp., Propionbacterium acnes, Anaerococcus spp., Candida spp., Escherichia coli, Malassezia pachydermatis, and Sphingomonas paucimobilis.

### BRIEF DESCRIPTION OF DRAWINGS

The accompanying drawings illustrate a disclosed embodiment and serves to explain the principles of the disclosed embodiment. It is to be understood, however, that the drawings are designed for purposes of illustration only, and not as a definition of the limits of the invention.

FIG. 1

FIG. 1 is a schematic illustration of the preparation of the pharmaceutical formulation in spray bottles as disclosed herein, with the delivery of 0.10 to 0.14 mL/spray, wherein weighted citric acid (100) and sodium citrate (200) are added to beaker 1 (400) containing purified water (300) to make a buffer solution. Thereafter, glycerine (500) and propylene glycol (600) are added to beaker 1 (400) containing the buffer solution and thoroughly mixed. The resultant mixture in beaker 1 (400) is added to beaker 2 (800) containing isopropanol (700) before the combined mixture is used to dissolve glycopyrronium bromide (900). The final volume of the pharmaceutical formulation in beaker 2 (800) is adjusted 100% (v/v) using purified water (1000) before packaging into individual spray bottles (1100) as samples for human and animal studies.

### EXAMPLES

Non-limiting examples of the invention will be further described in greater detail by reference to specific Examples, which should not be construed as in any way limiting the scope of the invention.

### Materials and Methods

All the reagents were obtained from commercial suppliers as shown in Table 1 and used without further purification.

### Example 1: Preparation of the pharmaceutical formulation

A pharmaceutical formulation was prepared using the following chemical components in the indicated amounts in Table 1.

TABLE 1
Composition of the pharmaceutical formulation

| Chemicals | Concentration | | Commercial Suppliers |
|---|---|---|---|
| Glycopyrronium | 2% | (w/v) | PCAS, Finland |
| bromide powder | | | |
| Citric acid | 0.32% | (w/v) | Medisca, USA |
| Sodium citrate | 0.084% | (w/v) | Medisca, USA |
| Glycerine | 10% | (v/v) | ICM Pharma Pte. Ltd., |
| | | | Singapore |
| Propylene glycol | 10% | (v/v) | ICM Pharma Pte. Ltd., |
| | | | Singapore |
| Isopropanol | 65% | (v/v) | Merck KGaA, Germany |
| Sterile water | ~15% | (v/v) | B. Braun, Germany |
| (make up formulation | | | |
| to 100% (v/v)) | | | |

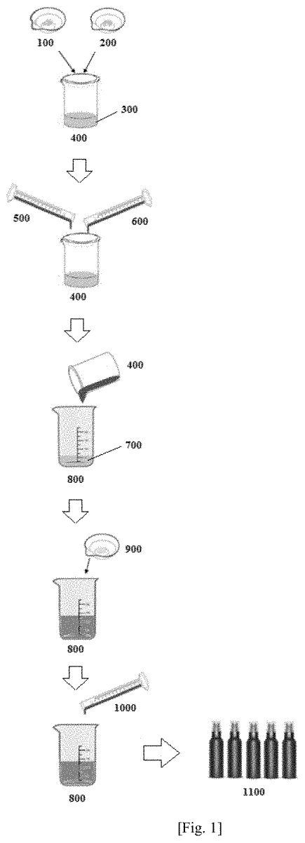

A schematic of the procedure to prepare the pharmaceutical formulation is as shown in FIG. 1. Briefly, glycopyrronium bromide powder (12 g), citric acid (1.92 g) and sodium citrate (0.504 g) were weighted separately on weighing paper. Citric acid and sodium citrate were dissolved in some purified water (70 ml) to make a buffer solution. Glycerine (60 ml) and propylene glycol (60 ml) were added to the buffer solution and mixed well by stirring. Isopropanol (390 ml) was added to the mixture to create the ternary solvent before using it to dissolve the anticholinergic agent glycopyrronium bromide powder.

The final volume of the formulation was adjusted to 100% (v/v) using purified water. The pharmaceutical formulation was packed into individual bottles and labelled as samples for the human and animal studies in Example 2 and 3 respectively.

### Example 2: Human studies using the pharmaceutical formulation

The pharmaceutical formulation as prepared in Example 1 was used by a confidential test group of five persons reported to suffer from bromhidrosis as a preliminary evaluation of the formulation efficacy on humans. The profile and the comparative results of the test group before and after use of the pharmaceutical formulation is as indicated in Table 2.

The severity of the sweating (hyperhidrosis) as self-reported by the test group was rated on a scale of 1 to 4 (HDSS, hyperhidrosis severity scale), 1 being the least severe and 4 being the most severe. The percentage reduction of bromhidrosis after use of the pharmaceutical formulation was as self-reported by the test group and the percentage mean reduction of bromhidrosis was calculated.

TABLE 2
Profile of human subjects and comparative results before
and after use of the pharmaceutical formulation

| | H1 | H2 | H3 | H4 | H5 |
|---|---|---|---|---|---|
| Age (years) | 51 | 24 | 34 | 46 | 22 |
| Gender | Female | Male | Male | Male | Male |
| Race | Indian | Indian | Chinese | Chinese | Chinese |
| Prior | Aluminium | Aluminium | Aluminium | Aluminium | Aluminium |
| treatment(s) | salt anti- | salt anti- | salt anti- | salt anti- | salt anti- |
| | perspirants | perspirants | perspirants | perspirants | perspirants |
| | (caused itch | (ineffective) | (ineffective) | (caused | (caused hot |
| | and was | and antiseptic | | itch, hence | sensation) |
| | ineffective) | washes | | stopped | |
| | and antiseptic | | | treatment) | |
| | washes | | | | |

Before Treatment with Pharmaceutical Formula

| | | | | | |
|---|---|---|---|---|---|
| Severity of | 2 | 1 | 2 | 2 | 3 |
| sweating (HDSS | | | | | |
| scale of 1 to 4) | | | | | |
| Descriptive | Distressed | Distressed | Bothered | Bothered | Not |
| severity of | | | | | bothered |
| bromhidrosis | | | | | |
| (not bothered, | | | | | |
| bothered and | | | | | |
| distressed) | | | | | |

| | |
|---|---|
| Dosage regime | The pharmaceutical formulation is sprayed on the axilla 1 or 2 |
| | sprays at a frequency of once in the morning and optionally once |
| | in the afternoon depending on person's requirement. |

Two Months After Treatment with Pharmaceutical Formulation

| Severity of | 1 | 1 | 1 | 2 | 2 |
|---|---|---|---|---|---|
| sweating (HDSS | | | | | |
| scale of 1 to 4) | | | | | |
| Reduction of | 50 | N.A. | 80 | 0 | 30 |
| sweatiness (%) | | | | | |
| Descriptive | Not | Not | Not | Bothered | Not |
| severity of | bothered | bothered | bothered | | bothered |
| bromhidrosis | | | | | |
| (not bothered, | | | | | |
| bothered and | | | | | |
| distressed) | | | | | |
| Reduction of | 100% | 100% | 100% | 15% | 50% |
| bromhidrosis (%) | | | | | |
| Mean reduction | 73% | | | | |
| of bromhidrosis (%) | | | | | |
| Side effects | None | None | None | None | None |

Based on the results in Table 2, it is shown that the pharmaceutical formulation is effective in reducing bromhidrosis in the test group with a 73% overall reduction of bromhidrosis. There was also no irritation and adverse reactions reported on the human body. The pharmaceutical formulation does not case dryness or darkening of the skin.

### Example 3: Animal studies using the pharmaceutical formulation

The pharmaceutical formulation as prepared in Example 1 was applied on a Shetland sheepdog with bromhidrosis as a preliminary evaluation of the formulation efficacy on dogs. The profile and the comparative results of the test dog before and after use of the pharmaceutical formulation is as indicated in Table 3.

TABLE 3
Profile and comparative results of the test dog before
and after use of the pharmaceutical formulation

| | A1 |
|---|---|
| Species | Shetland sheepdog |
| Gender | Male |
| Age (years) | 1 |
| Prior treatment(s) | None |

Before Treatment with Pharmaceutical Formulation

| | |
|---|---|
| Owner's perception of odour | 8 |
| severity (scale of 1 to 10) | |
| Dosage regime | Sprayed on affected skin 1 to 3 |
| | times a day, at 4 to 8 hours |
| | between sprays |

After Treatment with Pharmaceutical Formulation

| | |
|---|---|
| Onset of effect | 3 days |
| Owner's perception of odour | 3 |
| severity (scale of 1 to 10) | |
| Reduction of bromhidrosis (%) | 62.5% |
| Side effects | None |

Based on the results in Table 3, it is shown that the pharmaceutical formulation is effective in reducing bromhidrosis in the test dog with a 62.5% % reduction of bromhidrosis. There was also no irritation and adverse reactions reported for the test dog.

### INDUSTRIAL APPLICABILITY

The pharmaceutical formulation may be used as a treatment for pathological or non-pathological body malodour in humans and dogs. The pharmaceutical formulation is applicable in various applications, but not limited to, consumer care, healthcare and cosmetics industries.

It will be apparent that various other modifications and adaptations of the invention will be apparent to the person skilled in the art after reading the foregoing disclosure without departing from the spirit and scope of the invention and it is intended that all such modifications and adaptations come within the scope of the appended claims.

1. A pharmaceutical formulation comprising an anticholinergic agent and a ternary solvent comprising 5% to 15% v/v glycol, 60% to 70% v/v alcohol and 5% to 20% v/v water.

2. The pharmaceutical formulation of claim 1, wherein the anticholinergic agent is selected from the group consisting of glycopyrronium bromide, hyoscyamine, atropine, scopolamine, benzatropine, clidinium bromide, cyclopentolate, darifenacin, dicylomine, fesoterodine, homatropine hydrobromide, ipratropium, orphenadrine, oxybutynin, propantheline, methscopolamine, solifenacin, tiotropium, tolterodine, trihexphenidyl, trospium and mixtures thereof.

3. The pharmaceutical formulation of claim 1 or 2, wherein the alcohol is selected from the group consisting of ethanol and isopropanol.

4. The pharmaceutical formulation of any one of claims 1 to 3, wherein the glycol is selected from the group consisting of propylene glycol, butylene glycol and pentylene glycol.

5. The pharmaceutical formulation of any one of claims 1 to 4, wherein the anticholinergic agent is in an amount of 1.0% w/v, 2.0% w/v, 3.0% w/v, 4.0% w/v, 5.0% w/v or 6.0% w/v of the pharmaceutical formulation.

6. The pharmaceutical formulation of any one of claims 1 to 5, wherein the concentrations of the components in the ternary solvent are:
glycol at 1.0% w/v, 2.0% w/v, 3.0% w/v, 4.0% w/v, 5.0% w/v or 6.0% w/v of the pharmaceutical formulation;
alcohol at 60.0% v/v, 61.0% v/v, 62.0% v/v, 63.0% v/v, 64.0% v/v, 65.0% v/v, 66.0% v/v, 67.0% v/v, 68.0% v/v, 69.0% v/v or 70.0% v/v of the pharmaceutical formulation; and
water at 5.0% v/v, 6.0% v/v, 7.0% v/v, 8.0% v/v, 9.0% v/v, 10.0% v/v, 11.0% v/v, 12.0% v/v, 13.0% v/v, 14.0% v/v, 15% v/v, 16% v/v, 17% v/v, 18% v/v, 19% v/v or 20.0% v/v of the pharmaceutical formulation.

7. The pharmaceutical formulation of any one of claims 1 to 6, wherein when the anticholinergic agent is glycopyrronium bromide and the alcohol is isopropanol, the ratio of the components in the ternary solvent is such that the ratio of propylene glycol: isopropanol: water is 2:13:2.5 v/v.

8. The pharmaceutical formulation of any one of claims 1 to 7, further comprising an excipient material suitable for topical administration.

9. The pharmaceutical formulation of claim 8, wherein the excipient material is selected from the group consisting of anti-bacterials, anti-fungals, humectants, emulsifiers, preservatives, dispersants, emollients, surfactants, structurants, absorption promoters, antiseptics, anaesthetics, keratolytics, wound healing agents, lubricants, anti-perspirants, depilatories, UV-protectants, anti-inflammatories, steroids, antioxidants, antihistamines, skin and hair conditioners, fragrances, essential oils and natural plant extracts.

10. The pharmaceutical formulation of claim 9, wherein the humectant is selected from the group consisting of glycerine, glycerol, lecithin, gelatin, lactic acid, hyaluronic acid, glyceryl triacetate, hexylene glycol, butylene glycol, sorbitol, allantoin, sodium hyaluronate, sodium lactate, ammonium lactate, sodium pyrrolidine and urea.

11. The pharmaceutical formulation of any of one of claims 1 to 10, wherein said pharmaceutical formulation is for topical use.

12. The pharmaceutical formulation of any of one of claims 1 to 11, wherein said pharmaceutical formulation is antichlolinergic and antimicrobial or antichlolinergic and antibacterial.

13. The pharmaceutical formulation of any of one of claims 1 to 12, wherein said pharmaceutical formulation is the form selected from the group consisting of solution, lotion, cream, ointment, gel, paste, aerosol, foam and spray.

14. The pharmaceutical formulation of any of one of claims 1 to 13, wherein when the pharmaceutical formulation is in the form of a spray for use in human, the pharmaceutical formulation is sprayed on the axilla 1 or 2 sprays per administration at a frequency of at least once in a day, wherein once in the morning and optionally once in the afternoon, depending on a person's requirement.

15. The pharmaceutical formulation of any of one of claims 1 to 13, wherein when the pharmaceutical formulation is in the form of a spray for use in dog, the pharmaceutical formulation is sprayed on the affected skin at a frequency of 1 to 3 times a day and 4 to 8 hours between sprays.

16. The pharmaceutical formulation of any of one of claims 1 to 15, wherein when the pharmaceutical formulation is in the form of a spray, the amount of anticholinergic agent in each spray administered is 2.00 mg, 2.10 mg, 2.20 mg, 2.30 mg, 2.40 mg, 2.50 mg, 2.60 mg, 2.70 mg or 2.80 mg.

17. A method of preparing the pharmaceutical formulation of any one of claims 1 to 16, comprising the step of mixing the anticholinergic agent to a mixture containing alcohol, glycol, water, humectant and pH buffering agent at room temperature.

18. The method of claim 17, wherein said alcohol is selected from the group consisting of ethanol and isopropanol.

19. The method of claim 17 or 18, wherein said pH buffering agent is selected from the group consisting of organic acids, amino acids, polyaminocarboxylic acids, citrate salts, polyphosphates and combinations thereof.

20. The method of any one of claims 17 to 19, wherein said pH buffering agent is selected from the group consisting of citric acid, acetic acid, tartaric acid, ethylenediaminetetraacetic acid, 2,3-dimercaptopropanesulfonic acid, thiamine tetrahydrofurfuryl acid, alpha lipoic acid, glycine, sodium citrate, potassium citrate, sodium phosphate and combinations thereof.

21. The method of any one of claims 17 to 20, wherein said humectant is in an amount of 5.0% w/v, 6.0% w/v, 7.0% w/v, 8.0% w/v, 9.0% w/v, 10.0% w/v, 11.0% w/v, 12.0% w/v, 13.0% w/v, 14.0% w/v, 15.0% w/v, 16.0% w/v, 17.0% w/v, 18.0% w/v, 19.0% w/v or 20.0% w/v of the pharmaceutical formulation.

22. The method of any one of claims 17 to 21, wherein said pH buffering agent is in an amount in the range of 0.05% w/v to 1.00% w/v of the pharmaceutical formulation.

23. A method of inhibiting non-pathological body malodour in a mammal, comprising the step of administrating a pharmaceutical formulation to an area on said mammal, to inhibit apocrine secretion and inhibit the activity or prevent the overgrowth of the microorganism resident on said area, wherein said pharmaceutical formulation comprises an anticholinergic agent and a ternary solvent comprising 5% to 15% v/v glycol, 60% to 70% v/v alcohol and 5% to 20% v/v water.

24. The method of claim 23, wherein said mammal is selected from the group consisting of primates, caniformia and artiodactyla.

25. The method of claim 23 or 24, wherein said microorganism is selected from the group consisting of Corynebacterium spp., Corynebacterium minutissimum, Corynebacterium striatum, Corynebacterium jeikeium, Staphylococcus spp., Staphylococcus aureus, Staphylococcus epidermidis, Staphylococcus haemolyticus, Staphylococcus pseudintermedius, Streptococcus spp., Micrococcus spp, Propionbacterium spp., Propionbacterium acnes, Anaerococcus spp., Candida spp., Escherichia coli, Malassezia pachydermatis and Sphingomonas paucimobilis.

26. A method of treating apocrine secretion causing odour and reduction of microbial overgrowth in a mammalian subject, comprising administrating the pharmaceutical formulation to an area on said mammalian subject exhibiting said microbial overgrowth, wherein said pharmaceutical formulation comprises an anticholinergic agent and a ternary solvent comprising 5% to 15% v/v glycol, 60% to 70% v/v alcohol and 5% to 20% v/v water.

27. A pharmaceutical formulation for use in the treatment of apocrine secretion causing odour and reduction of microbial overgrowth in a mammalian subject, wherein said pharmaceutical formulation comprises an anticholinergic agent and a ternary solvent comprising 5% to 15% v/v glycol, 60% to 70% v/v alcohol and 5% to 20% v/v water.

28. Use of a pharmaceutical formulation, in the manufacture of a medicament for the treatment of apocrine secretion causing odour and reduction of microbial overgrowth in a mammalian subject, wherein said pharmaceutical formulation comprises an anticholinergic agent and a ternary solvent comprising 5% to 15% v/v glycol, 60% to 70% v/v alcohol and 5% to 20% v/v water.

29. The method of claim 26 or the pharmaceutical formulation for use of claim 27 or the use of claim 28, wherein said mammalian subject is selected from the group consisting of primates, caniformia and artiodactyla.

30. The method of claim 26 or the pharmaceutical formulation for use of claim 27 or the use of claim 28, wherein said microbial overgrowth is caused by a microorganism selected from the group consisting of Corynebacterium spp., Corynebacterium minutissimum, Corynebacterium striatum, Corynebacterium jeikeium, Staphylococcus spp., Staphylococcus aureus, Staphylococcus epidermidis, Staphylococcus haemolyticus, Staphylococcus pseudintermedius, Streptococcus spp., Micrococcus spp, Propionbacterium spp., Propionbacterium acnes, Anaerococcus spp., Candida spp., Escherichia coli and Malassezia pachydermatis.
